(19) United States
(12) Patent Application Publication
(10) Pub. No.: US 2023/0368223 A1
(43) Pub. Date: Nov. 16, 2023

(54) METHOD AND DEVICE FOR DATA PROCESSING BASED ON DATA VALUE

(71) Applicant: Peking University, Beijing (CN)

(72) Inventors: Jie SONG, Beijing (CN); Yue ZHAO, Beijing (CN); Yanzhi WANG, Beijing (CN)

(21) Appl. No.: 18/314,899

(22) Filed: May 10, 2023

(30) Foreign Application Priority Data
May 11, 2022 (CN) 202210532391.6

(51) Int. Cl. G06Q 30/0202

(52) U.S. Cl. CPC G06Q 30/0202

(57) ABSTRACT

A computer-implemented method for data processing based on a data value includes: calculating a value of each piece of original data based on a utility function associated with a service revenue; acquiring pieces of high-value data from the original data based on the value of each piece of original data; and performing a service prediction on the pieces of high-value data.

### CROSS-REFERENCE TO RELATED APPLICATIONS

This application claims priority to Chinese Patent Application Serial No. 202210532391.6, filed on May 11, 2022, the entire disclosure of which is incorporated herein by reference.

### TECHNICAL FIELD

The present disclosure relates to processing massive data, and more specifically, relates to a method and a device for data processing based on a data value, and a storage medium.

### BACKGROUND

Various industries are undergoing a digital transformation. Data is used by a digital platform to perform an intelligent service decision. However, big data results in problems such as a high calculation cost and a lack of a gripper in data management.

Full data are usually utilized due to a lack of effective data management, which are not only limited to computing resources but also are affected by noise data.

For example, an online medical platform desires to establish a consultation demand prediction model for each doctor, so as to optimize a resource configuration. If the full data are used to train the model, the calculation cost is relatively high. In addition, an influence of noise data such as click farming exists on the online medical platform. That is, some doctors encourage patients to perform false evaluations or click articles and gifts on the platform in order to obtain a higher return.

However, it is difficult to evaluate a value of a data point in the related art, and it is unable to manage the data in a targeted manner, which causes the use efficiency of data is not high.

### SUMMARY

According to a first aspect of the present disclosure, a computer-implemented method for data processing based on a data value is provided. The method includes: calculating a value of each piece of original data based on a utility function associated with a service revenue; acquiring pieces of high-value data from original data based on the value of each piece of original data; and performing a service prediction on the acquired high-value data.

According to a second aspect of the present disclosure, a computing device is provided, which includes a processor, and a memory stored with instructions executable by the processor. The processor is configured to: calculate a value of each piece of original data based on a utility function associated with a service revenue; acquire pieces of high-value data from original data based on the value of each piece of original data; and perform a service prediction based on the acquired high-value data.

According to a third aspect of the present disclosure, a non-transitory computer readable storage medium having computer programs stored thereon is provided. When the computer programs are executed by a computing device, a method for data processing based on a data value is implemented. The method includes: calculating a value of each piece of original data based on a utility function associated with a service revenue; acquiring pieces of high-value data from original data based on the value of each piece of original data; and performing a service prediction on the acquired high-value data.

### BRIEF DESCRIPTION OF THE DRAWINGS

FIG. 1 is a block diagram illustrating an apparatus for data processing according to an exemplary embodiment of the present disclosure.

FIG. 2 is a block diagram illustrating an estimation unit according to an exemplary embodiment of the present disclosure.

FIG. 3 is a flowchart illustrating a method for data processing according to an exemplary embodiment of the present disclosure.

FIG. 4 is a block diagram illustrating a prediction system for an online medical platform according to an exemplary embodiment of the present disclosure.

FIG. 5 illustrates a correlation between different doctor information variables.

FIG. 6 illustrates a relationship between a platform revenue and a prediction error, a relationship between an extra cost of a platform and a prediction error.

FIG. 7 illustrates a change in a model performance after gradually removing data.

### DETAILED DESCRIPTION

The present disclosure may be more readily understood based on the following description of attached drawings, in which same numerals indicate the same structure units.

According to the following embodiments of the present disclosure, an intelligent service decision may be made based on historical data in a service system, since high-value data are screened out from a large amount of historical data, and a service prediction is performed on the basis of the screened high-value data. In this way, an operational burden of the service system may be reduced, and the accuracy of service prediction may be improved.

According to an exemplary embodiment of the present disclosure, a service revenue is used as a basis for measuring the data value, so that a service improvement is effectively associated with the data value, which may more effectively screen out the data that helps improve a decision-making performance of the service platform and facilitate to interpret a decision-making mechanism.

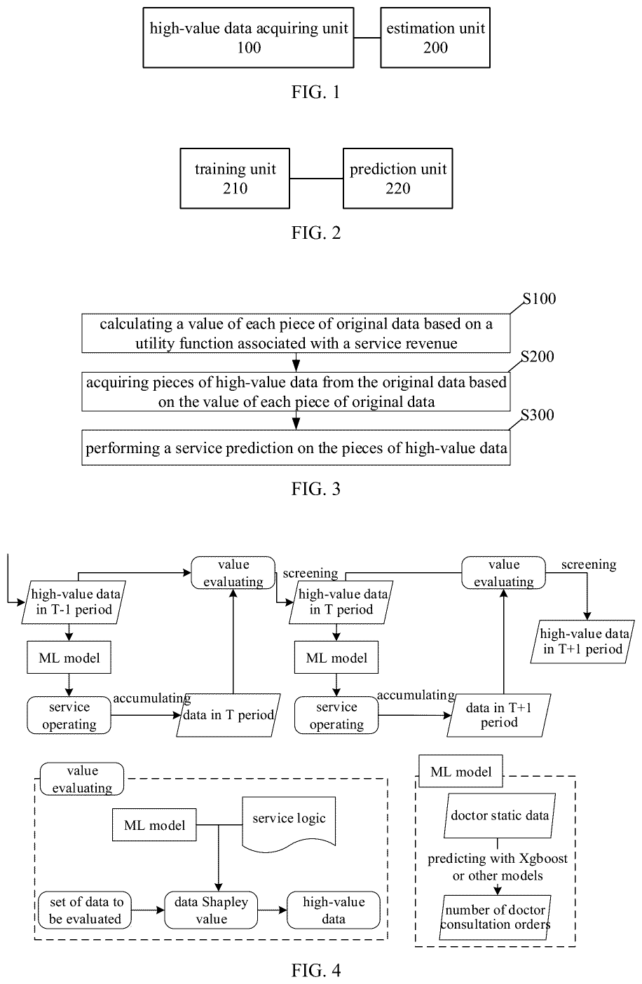

As an example, FIG. 1 illustrates a block diagram of an apparatus for data processing according to an exemplary embodiment of the present disclosure. An apparatus 10 for data processing as illustrated in FIG. 1 includes a high-value data acquiring unit 100 and an estimation unit 200.

As illustrated in FIG. 1, the high-value data acquiring unit 100 is configured to acquire at least a portion of high-value data from original data.

The original data may be data that is generated online, data that is generated and stored in advance, or data that is received from outside via an input device or a transmission medium. The data may relate to attribute information of an individual, an enterprise or an organization, such as an identity, an education, an occupation, an asset, a contact, a liability, an income, a revenue and a tax payment. Alternatively, the data may also relate to other attribute information of service-related items, for example, information about a transaction amount of a sales contract, both parties of the sales transaction, the object of transaction and a transaction place. It should be noted that data contents mentioned in the exemplary embodiment of the present disclosure may relate to a performance or property of any object or transaction in a certain aspect of the service, which is not limited to defining or describing an individual, an object, an organization, a unit, an institution, an item and an event.

The original data may be structured data or unstructured data from different sources, for example, text data or numerical data. The data may be derived from an internal entity that is expected to perform a service prediction, for example, a bank, an enterprise, a school, etc. from which a prediction result is expected to be acquired. The data may also be derived from an external entity such as a data provider, the Internet (for example, a social website), a mobile operator, an APP operator, an express company, a credit institution, etc. Optionally, the internal data and the external data may be used in combination to form original data carrying more information.

The original data may be input into the high-value data acquiring unit 100 by an input device, or may be automatically generated by the high-value data acquiring unit 100 based on existing data, or may be acquired by the high-value data acquiring unit 100 from a network (for example, a storage medium on the network (for example, a data warehouse)). In addition, an intermediate data exchange device such as a server may help the high-value data acquiring unit 100 acquire corresponding data from an external data source. A data conversion module such as a text analysis module in the high-value data acquiring unit 100 may convert the acquired data into a format that is easy to be processed. It should be noted that the high-value data acquiring unit 100 may be configured as various modules consisting of a software, a hardware and/or a firmware, some or all of which may be integrated or may work together to complete a particular function.

According to an exemplary embodiment of the present disclosure, the high-value data acquiring unit 100 may calculate a value of each piece of original data based on a utility function associated with a service revenue, and acquire at least a portion of the high-value data based on a calculation result. When the high-value data acquiring unit 100 screens out the high-value data, unification of data quality and service operation is achieved by taking the service revenue as a measurement index, so that the prediction performance may be improved more effectively while the operational burden is reduced.

In addition, the apparatus 10 for data processing as illustrated in FIG. 1 may further include a value display unit (not shown). The value display unit is configured to build a graphical display interface based on the acquired high-value data to interpret the service model to a user. The value display unit may draw a graphical display interface, such as a graph, based on the acquired high-value data, alone or in combination with other related data, to help the user view the function/role of the high-value data in the model, and understand the service logic better.

As illustrated in FIG. 1, the estimation unit 200 is configured to perform a service prediction based on the acquired high-value data. The estimation unit 200 may perform the service prediction on high-value data based on various ways. For example, corresponding rules are generated based on statistical properties presented by the high-value data, and future data are then predicted based on these rules.

According to an exemplary embodiment of the present disclosure, the original data may include at least one period of historical data that occurs before a current moment, and correspondingly, the estimation unit 200 performs the service prediction for a next period of data based on the acquired high-value data, to obtain prediction data corresponding to the current moment.

In addition, the apparatus 10 for data processing illustrated in FIG. 1 may further include a feedback unit (not shown). The feedback unit is configured to collect a real service result corresponding to the prediction data, to form a newly added historical data sample. Then, high-value data is acquired again based on the updated historical data, and so on, this cycle forms a data loop of the service operation system.

In the present disclosure, the estimation unit 200 may make an intelligent decision based on the high-quality data. For example, a model (for example, a machine learning model such as an artificial intelligence (AI) model) is trained by using high-quality data, and the service prediction is performed by using the trained model.

FIG. 2 is a block diagram illustrating an estimation unit according to an exemplary embodiment of the present disclosure. As an example, the estimation unit 200 includes a training unit 210 and a prediction unit 220.

The training unit 210 is configured to train a service model by using the acquired high-value data. As an example, the training unit may train a machine learning model by using the acquired high-value data. Machine learning (ML) is an inevitable product of AI development to a certain stage, for improving the performance of a system by learning the experience via calculation. In a computer system, “experience” usually exists in the form of “data”, and a “model” may be generated from the data based on a machine learning algorithm, that is, empirical data are provided to the machine learning algorithm and a model is generated based on the empirical data. When a new situation is faced, the model may provide a corresponding determination, that is, a prediction result. Machine learning may be implemented as “supervised learning”, “unsupervised learning” or “semi-supervised learning”. It should be noted that, a particular machine learning algorithm is not limited by the exemplary embodiments of the present disclosure. In addition, it should also be noted that in a process of training and applying a model, other means such as a statistical algorithm may also be combined.

The prediction unit 220 may perform the service prediction by using a service model trained by the training unit 210. The model may be trained by the training unit 210 offline or online. The training not only includes a first training of the model, but also an update of the model. Accordingly, the prediction unit 220 may perform an offline or online prediction by using the trained service model, which is not limited in the exemplary embodiments of the present disclosure.

The apparatus illustrated in FIG. 1 and FIG. 2 may be respectively configured as a software, a hardware, a firmware, or any combination of the forgoing, that performs specific functions. For example, each apparatus may correspond to a dedicated integrated circuit, or pure software codes, and units or modules with a combination of hardware and software. In addition, one or more functions implemented by each apparatus may also be performed uniformly by components in a physical entity device (such as a processor, a client, or a server, etc.).

The method for data processing according to an exemplary embodiment of the present disclosure is described referring to FIG. 3.

As an example, the method illustrated in FIG. 3 may be performed by the apparatus illustrated in FIG. 1, or may be completely implemented by means of a software via a computer program, and may further be performed by a computing device with a specific configuration. For ease of description, assuming that the method illustrated in FIG. 3 is performed by the apparatus illustrated in FIG. 1.

At step S100, a value of each piece of original data is calculated based on a utility function associated with a service revenue.

As an example, the high-value data acquiring unit 100 may collect the original data in a manual, semi-automatic, or full-automatic mode, or process the collected original data, so that the processed data has an appropriate format or form. As an example, the high-value data acquiring unit 100 may collect the original data in batches.

The high-value data acquiring unit 100 may receive original data records manually input by a user by means of an input device (for example, a workstation). In addition, the high-value data acquiring unit 100 may take out the original data records from a data source system in a fully automatic mode. For example, a data source is systematically requested and the requested original data is obtained from the response by a timer mechanism implemented in a software, a firmware, a hardware, or a combination thereof. The data source may include one or more databases or other servers. Full-automatic data acquisition may be implemented via an internal network and/or an external network, in which encrypted data transmitted over the Internet may be included. When a server, a database and a network are configured to communicate with each other, data acquisition may be automatically performed without manual intervention. However, it should be noted that a certain user input operation may still be present in this manner. A semi-automatic mode is between a manual mode and a full-automatic mode. The difference between the semi-automatic mode and the full-automatic mode lies in that a trigger mechanism activated by a user replaces the above timer mechanism. In this case, a request for extracting data is generated only when a particular user input is received. In an example, when the original data are acquired each time, the captured data may be stored in a non-volatile memory. As an example, a data warehouse may be configured to store original data collected during acquisition, and processed data.

The acquired original data records may be derived from the same or different data sources, that is, each data record may also be a splicing of different data records. For example, in addition to acquiring an information data record (which includes attribute information fields such as income, education, post and asset conditions) filled when a client applies for signing up a credit card to a bank, as an example, the high-value data acquiring unit 100 may further acquire other data records of the client in the bank, such as loan record data and daily transaction data. The acquired data records may be spliced into a complete data record. In addition, the high-value data acquiring unit 100 may further acquire data derived from other private sources or public sources, for example, data derived from a data provider, data derived from the Internet (e.g., a social website), data derived from a mobile operator, data derived from an APP operator, data derived from an express company and data derived from a credit institution.

Optionally, the high-value data acquiring unit 100 may store and/or process collected data by means of a hardware cluster (such as a Hadoop cluster, a Spark cluster, etc.). The processing may be, for example, storage, classification and other offline operations. In addition, the high-value data acquiring unit 100 may also perform online streaming processing on the collected data.

As an example, the high-value data acquiring unit 100 may include a data conversion module such as a text analysis module, and correspondingly, at step S100, the high-value data acquiring unit 100 may convert unstructured data (such as a text) into structured data that is easy to use for subsequent processing or reference. Text-based data may include an email, a document, a web page, a graph, an electronic data table, a call center log, and a transaction report.

As an example, the exemplary embodiments of the present disclosure may use service data related to at least one of the following items: image recognition, e.g., optical character recognition (OCR), face recognition (security), object recognition (a traffic sign), and picture classification; speech recognition, e.g., natural language processing fused in a voice assistant; natural language processing, e.g., examining a text (e.g., a contract, a legal document, a customer service record), spam recognition, text classification (emotion, intention, theme); automatic control, e.g., automatic control on energy industries (a mine, a wind turbine generator system), energy saving (an air conditioning system); intelligent question answering, e.g., a chatbot, or an intelligent customer service, operation decision-makings (e.g., financial science and technology, including marketing and customer obtaining, anti-fraud, anti-money laundering, underwriting and credit scoring, etc.), medical treatment (e.g., disease screening and prevention, personalized health management, auxiliary diagnosis), municipal administration (e.g., social governance and supervision law enforcement, resource environment and facility management, industry development and economic analysis, public service and livelihood guarantee, smart city, etc.), and recommendation services (e.g., advertisement, consultation, music, videos, financial products (financing, insurance).

After the original data are acquired, the high-value data acquiring unit 100 may calculate the value of each piece of original data based on the utility function associated with the service revenue.

In an exemplary embodiment of the present disclosure, the high-value data acquiring unit 100 calculates a data value based on the utility function related to the service revenue. The high-value data acquiring unit 100 may construct a calculation method of a service revenue based on a period of service prediction, analyze each component in the service revenue, extract a portion related to data contribution, and construct the utility function for calculating the data value based on the extracted portion related to data contribution. As an example, the high-value data acquiring unit 100 may calculate a value of the original data based on a data Shapley value. Specifically, the high-value data acquiring unit 100 may calculate the data Shapley value of each piece of original data based on the utility function associated with the service revenue as the value of each piece of original data.

At step S200, pieces of high-value data are acquired from the original data based on the value of each piece of original data.

At step S300, the service prediction is performed based on the pieces of high-value data.

In an embodiment, the estimation unit 200 may perform a prediction only based on the high-value data screened out. As an example, a service model may be trained by the training unit 210 in the estimation unit by using the acquired high-value data. The high-value data used may only include high-value data acquired in a current period, or may include all high-value data acquired historically, or may be any periods or some periods of high-value data screened. As an example, the training unit 210 may train the service model by using any applicable machine learning algorithm. Then, the prediction unit 220 in the estimation unit 200 performs the service prediction by using the service model trained by the training unit 210. The prediction unit 220 may respectively input all the data to be predicted into the service model, to acquire a prediction result corresponding to each piece of data to be predicted. Further, a corresponding service decision may be made based on the prediction result, for example, service resource allocation.

Exemplary embodiments of the present disclosure are described below with an online medical platform as an example. However, it should be understood that the exemplary embodiments of the present invention are not limited to the online medical platform, but are applicable to any similar service prediction system, such as a prediction of airport passenger flow distribution, a music trend prediction, a demand prediction and storage planning solution, a prediction of Sina Weibo interaction volume, a prediction of monetary fund inflow and outflow, a movie box office prediction, a prediction of agricultural product price, a prediction of Tibetan Plateau lake area based on multi-source data, a prediction of microblog propagation scale and propagation depth, an abalone age prediction, a prediction of student performance ranking, a travel flow prediction of online car-hailing, a prediction of red wine quality score, a prediction of search amount and stock price fluctuation of a search engine, a prediction of rural resident income growth, a real estate sales influence factor analysis, a prediction of stock price trend, a prediction of national comprehensive transportation total amount and an earthquake prediction.

According to an exemplary embodiment of the present disclosure, a service model may relate to an online medical platform, and correspondingly, a service prediction is performed to predict a number of treatments that each doctor may undertake in a next period based on attribute information of each doctor on the online doctor medical platform. FIG. 4 is a block diagram illustrating a prediction system for an online medical platform according to an exemplary embodiment of the present disclosure.

In the rapid development of digital economy, traditional medical resources are combined with a digital platform to generate an emerging medical mode—an online medical platform. Compared with a traditional hospital, the online platform eliminates a spatial distance between medical resources and patients, and provides more treatment options for the patients in an era of COVID-19. Moreover, the platform may fully integrate and utilize resources by using the platform data, which greatly improves the efficiency of allocating the medical resources. For example, the online medical platform accumulates a large amount of data during its operation, and improves the platform efficiency by making a personalized recommendation and demand matching based on data, to acquire more data, thereby constituting a “virtuous cycle” of data-operation.

A typical service mode of the platform is as follows: a patient retrieves a desired doctor on the platform in combination with disease symptoms of the patient, and selects his/her favorite doctor for consultation based on doctor static data in a doctor list returned by the platform. For example, for a recommended doctor in “hypertension” diseases retrieved by a patient, the patient selects an appropriate doctor for consultation based on doctor homepage information and data such as “doctor gender”, “age”, “gift” and “thank-you note”.

The online medical platform fuses data with the traditional medical industry, which injects new power into the traditional industry. However, a problem of platform data governance is also increasingly concerned. On one hand, since big data describes medical behaviors on a platform more clearly, the platform achieves precise marketing with a finer granularity. However, the platform also needs to balance the data calculation cost and the effectiveness. On the other hand, there is also a “data click farming” phenomenon in the platform (such as malicious click farming, unreal evaluation), resulting in distortion of data. Therefore, it is very important for the platform to perform an appropriate value evaluation on data and screen out high-value data. In this way, the data calculation cost is reduced without reducing the algorithm performance, and a good platform ecology is constructed by screening out unreal data.

There is still a challenge to evaluate the data value in the related art. Determination of a data value may also facilitate data fusion, which provides a basis for data governance. The current research about the data value is mainly based on the perspective of economics from top to bottom, and a main measurement method includes a cost method, a market method and an income method. The three methods are constrained by data regulation, an incomplete data market and difficult splitting of the data value, resulting in a large measurement deviation of the data value.

According to an exemplary embodiment of the present disclosure, on an online medical platform, a doctor is employed to complete an online consultation service. It is very important for a platform to properly allocate a doctor resource (signing a contract that needs a longer online time with a doctor in high demand, and vice versa). Therefore, it is necessary to estimate a demand of the doctor in the future by using the data. For example, a total demand (e.g., a number of consultation orders) of the doctor within a period of time is predicted by using machine learning based on static data accumulated in a period of time, such as, a doctor age, a gender, a hospital level, a gift, a thank-you note, a patient registration, a waiting duration and a patient vote. Accordingly, a decision is made (a doctor resource allocation). However, since the data amount used for prediction is large and there are noise data due to behaviors such as click farming and fake comment, the loss in both the precision and the calculation cost may be caused by simply using full data. In addition, a black box model used by a platform is complex and the platform fails to make an effective interpretation of the black box model. As an example, in a machine learning process, the value of the used data may be evaluated to provide a guidance for data management of the platform, which reduces a calculation cost of the platform, improves a learning progress, and interprets an underlying logic of the black box model. For explanation of the model, the high-value data reflects data with a significant model prediction effect, and reflects data distribution in a normal service mode. For example, a high consultation amount corresponds to a large number of thank-you notes. From the high-value data, it may be determined what data distribution helps the model obtain a good result.

For a data value evaluation framework that is updated online in an online medical platform illustrated in FIG. 4, time is discretized into 1, 2, . . . , T periods. The framework evaluates and updates the data accumulated during the platform operation in each period, which assists in platform operation. The complete update and evaluation frameworks for the high-value data in T−1 and T periods are illustrated in the flowchart.

A “prediction+operation” process of the platform is described. In order to reasonably allocate doctor resources, the platform predicts a demand to a doctor by using the doctor static data accumulated in a previous period, and the data used for prediction are illustrated below:

| X: doctor static data accumulated | | | | Y: consultation orders of a doctor |
|---|---|---|---|---|
| during T-2 to T-1 periods | | | | during T-1 to T periods |
| Gender | Age | . . . | Gift | Total number of |
| | | | | consultation orders |

The platform may decide how to allocate the current resources based on the prediction result, so as to further accumulate data.

First, it is necessary to combine operation of a platform with a prediction behavior of machine learning to construct a corresponding utility function.

For example, for a medical platform, it is assumed that that time is discrete and infinite, t∈{0, 1, . . . }, an average time of a doctor i∈{1, 2, . . . n} serving a patient τᵢ (τᵢ=τ), a revenue share acquired by the platform from one service is rᵢ>0. The platform decides a cooperation agreement that a doctor signs in a t+1 period based on historical data in a t period. An online service duration required in a t period is denoted as Sᵢₜ, and a salary per unit duration is denoted as wᵢ>0. The data collected by the platform during the t period is denoted as Xₜ, and a patient demand of the doctor may be denoted as a random variable Dᵢₜ. The doctor cannot serve a patient who requests for exceeding a working duration. Such patient may be served by the platform with the cost α>0. In the case, a total revenue of the platform during the t period is expressed as:

$${R}_{t} = {\sum }_{i = 1}^{n} [ {r}_{i} * min \{ \frac{{S}_{it}}{τ} , {D}_{it} \} - {w}_{i} {S}_{it} - α ( {D}_{it} - min \{ \frac{{S}_{it}}{τ} , {D}_{it} \} ) ] \qquad ( 3.1 )$$

It can be seen from the above equation that the more accurate the patient demand estimation of a single doctor by the platform, the more revenue the platform may obtain. Assuming that the platform estimates a next period demand of each doctor through machine learning based on the historical data, a cooperation agreement is established accordingly, and a working duration of the doctor in the next period is expressed as:

Sᵢₜ=τ [symbol] [Dᵢₜ| [symbol] ₜ₋₁]

where [symbol] ₜ₋₁=[{Xₖ}ₖ₌₀^{t-1}] is a set of all historical data acquired before the t period, which embodies a complete chain of “data→prediction→decision→value”.

Further, let

$${\hat{D}}_{it} = \frac{{S}_{it}}{τ} ,$$

ΔDᵢₜ=\hat{D}ᵢₜ−Dᵢₜ, the above equation (3.1) may be accordingly adjusted as follows.

$$Let {R}_{ti} = {r}_{i} * min \{ \frac{{S}_{it}}{τ} , {D}_{it} \} - {w}_{i} {S}_{it} - α ( {D}_{it} - min \{ \frac{{S}_{it}}{τ} , {D}_{it} \} ) ,$$

$$if \frac{{S}_{it}}{τ} = {D}_{it} , then {R}_{ti} = {r}_{i} {D}_{it} - {w}_{i} {S}_{it} = ( {r}_{i} - {w}_{i} τ ) {D}_{it} ,$$

$$let {R}_{ti}^{*} = ( {r}_{i} - {w}_{i} τ ) {D}_{it} ;$$

$$if \frac{{S}_{it}}{τ} \geq {D}_{it} , {R}_{ti} = {r}_{i} {D}_{it} - {w}_{i} {S}_{it} = {R}_{ti}^{*} - {w}_{i} τ ( Δ {D}_{it} ) ; if \frac{{S}_{it}}{τ} < {D}_{it} ,$$

$${R}_{ti} = {r}_{i} * \frac{{S}_{it}}{τ} - {w}_{i} {S}_{it} - α ( {D}_{it} - \frac{{S}_{it}}{τ} ) = {R}_{ti}^{*} - ( - {r}_{i} + {w}_{i} τ + α ) Δ {D}_{it} .$$

When the demand estimation of the platform has a deviation, the allocation of resources may be temporarily adjusted to make up for the error of demand prediction, and when the demand prediction of the platform has a large deviation, an extra salary may be paid to the doctor, thereby reducing the platform revenue. When the predicted demand is insufficient, the platform needs to temporarily and additionally adjust a resource allocation. Assuming that a cost of adjusting the resource allocation cannot bring a positive revenue for the platform, the platform may be forced to make a more accurate prediction in advance. At this time, it is obtained ∀i, wᵢτ+α<rᵢ, where wᵢ>0.

It is noted that a revenue loss of the platform is closely related to ΔDᵢₜ, that is, a predicted absolute error. Specifically, since the predicted error may lead to the revenue loss of the platform (R*ₜ−Rₜ∝MAE(D, \hat{D})), the platform may define a utility function by using the predicted mean absolute error (MAE). The utility function is shown as below:

$$V ( ) = M - M A E ( D , \hat{D} ) = M - \frac{1}{n} {\sum }_{i = 1}^{n} \vert [ {D}_{it} \vert ] - {D}_{it} \vert , ∀ ⊂ {∞}_{}$$

That is, according to the exemplary embodiments of the present disclosure, the utility function is associated with a predicted error of a service model trained by using at least a portion of original data. The accuracy of platform data represented in machine learning is associated with the platform value by using the utility function.

In addition, optionally, the high-value data acquiring unit according to the exemplary embodiments of the present disclosure, may construct different utility functions in a similar way. For example, the utility function associated with the service revenue may be adjusted based on at least one of a service crowd, a service logic, an external environment and a time change.

Next, the data value may be calculated based on the utility function. According to the exemplary embodiments of the present disclosure, a data Shapley value of each piece of original data may be calculated based on the utility function associated with the service revenue as the value of each piece of original data.

In particular, the Shapley value is proposed by Lloyd Shapley to address equitable distribution of cooperation revenues. The Shapley value from a theory in the Game Theory is determined based on a “marginal contribution” of a participant, and in a cooperative game, the marginal contribution may be regarded as an influence on the cooperation revenues after a participant engages in the game. The contribution of each participant may be calculated by traversing a combination of all participants, and a specific equation is as below:

$${φ}_{z} ( v ) = {\sum }_{U ⊆ K} \frac{\vert U \vert ! ( n - \vert U \vert ) !}{n !} [ v ( U ⋃ \{ z \} ) - v ( U ) ]$$

where K is a universal set, and n=|K|, i∈K, the utility function v represents revenues of the cooperative game, φ_{z}(v) is a contribution of an element z under the definition of the utility function v, that is, a Shapley value of the element z.

According to an exemplary embodiment of the present disclosure, z may be regarded as each data point or feature in the model, and v may be considered as a loss function or accuracy (such as a utility function) that measures a model effect. In this case, φ_{z}(v) is the contribution of the data point or feature z to the loss function or the accuracy of the entire model, the value evaluation mode retains completeness, fairness and additivity of the Shapley value. The value of the data point may be effectively extracted in a machine learning scenario.

It can be seen from the above equation that the calculation complexity of the Shapley value is exponential, and “utility” of the calculation model in each literation means re-training the model by using a data subset or a feature subset U, which leads to a great calculation burden when model learning based on a large amount of data.

For this purpose, optionally, the high-value data acquiring unit may perform random permutation on a set of all the original data in each iteration of calculating the utility function. When a difference between a utility function value of a set consisting of any piece of original data and previous elements before the any piece of original data and a utility function value of the set of all the original data is less than a preset threshold, the high-value data acquiring unit may remain a utility function value of the any piece of original data unchanged for a set consisting of the previous elements.

That is, the control of algorithm complexity is performed by simplifying data or feature permutation. For example, the Shapley value may be expressed in the data or feature permutation:

$${φ}_{i} ( v ) = \frac{1}{N !} {\sum }_{π ∈ Π ( K )} [ v ( {P}_{i}^{π} ⋃ \{ i \} ) - v ( {P}_{i}^{π} ) ]$$

where π∈Π(K) is a permutation of all elements in a universal set, and Pᵢ^{π} is a set consisting of elements before the element i in the permutation. In other words, Pᵢ^{π}={π[1], . . . , π[j]}, where π[j+1]={i}. First, random permutation on a set of elements is performed. Second, when scanning from the first element to the last element, it is defined that a difference between a utility of a set consisting of a certain element and its previous elements and a utility of the universal set is less than a certain threshold, a marginal contribution (utility) of the certain element to the set including previous elements before the certain element is defined as 0. Finally, a Shapley value of the element is updated by using an average value. A pseudo code is as follows:

```
Input: Train data S = {1, . . . , n}, learning algorithm [symbol] , performance
score V
Output: Shapley value of training points: ϕ₁, . . . , ϕₙ
Initialize ϕᵢ = 0 for i = 1, . . . , n and t = 0
while Convergence criteria not met do
  t ← t + 1
  πⁱ: Random permutation of train data points
  v₀^{t} ← V(∅, [symbol] )
  for j ∈ {1, . . . , n) do
    if |V (S) − vⱼ₋₁^{t}| < Performance Tolerance then
      vⱼ^{t} = vⱼ₋₁^{t}
    else
      vⱼ^{t} ← V({πⁱ[1], . . . , πⁱ[j]}, [symbol] )
    end if
    ${ϕ}_{{π}^{i} [ f ]} ← \frac{t - 1}{t} {ϕ}_{{π}^{i - 1} [ j ]} + \frac{1}{t} ( {v}_{j}^{i} - {v}_{j - 1}^{i} )$
  end for
end while
```

According to the method, if a threshold is satisfied, a marginal utility of a data node is regarded as 0, and each permutation of an element set is arbitrary. In addition, the method uses a property of the Shapley value, that is, under a certain assumption, as a data volume or a feature volume increases, a utility of the data set may be converged to the utility of the data or feature universal set, which may be expressed as

$${lim}_{i \to n} {v}_{i} = V ( D , A ) .$$

In other words, when a data volume is small, a contribution of the newly added data to the model is relatively high; and however, when the data volume is large, a contribution of the newly added data to the model may decrease and be close to 0.

In fact, an approximate Shapley value may be obtained within a shorter time by controlling a number of iterations and a threshold of the algorithm. Compared with the existing method of calculating the Shapley value, the calculation efficiency of the disclosure is improved to a certain extent.

As another example, considering that the performances of most machine learning models are continuous in a feature space, adjacent data points tend to have the same prediction results. Based on the property, the high-value data acquiring unit may calculate the data Shapley value of each piece of original data by: training a universal set service model based on the set of all the original data, recording a universal set prediction result of each piece of original data in the universal set service model, and in each iteration of calculating the utility function, acquiring a prediction result of the original data based on a universal set prediction result of at least one piece of original data close to the original data. In this way, it may be avoided a model is re-trained.

As an example, the model is trained based on a complete training set, and a prediction of each data point may be recorded. Further, when prediction results in a prediction set need to be calculated in each iteration, the predicted results may be estimated by using the averaging of K nearest predicted values of the training set. That is, for any data point (x, y)∈S′ in a test set S′, assuming that a K-nearest neighbor set of x in the training set is Dₓ, and a prediction model is f, then a predicted value yₚᵣₑ for y is expressed as:

$${y}_{pre} = \frac{1}{K} {\sum }_{{x}^{′} ∈ {D}_{x}} f ( {x}^{′} ) .$$

Finally, the utility function v is calculated based on all the approximate prediction results in the prediction set, and there is no need to re-train the model, so that a calculation time may be greatly shortened.

It should be understood that the foregoing two optimization processing manners may be used alone or in combination.

Assume that the online medical platform has about 20 million pieces of doctor data, and may acquire doctor data in the years of 2018, 2019 and 2020. In order to reduce a calculation complexity, 5000 pieces of doctor information and their corresponding consultation order information are extracted from the doctor data to conduct a verification research. A model for predicting demands of a doctor in next year is constructed by utilizing doctor static information and platform behavior information, to research the value of the doctor data accordingly.

Based on data characteristics, the selected doctor data may be divided into the following two types. A first one is doctor registration information, which is information provided by a doctor when the doctor registers on an Internet medical platform, including gender, title, hospital level of the doctor. This information reflects a consultation capability that the doctor statically provides. A second type is doctor platform behavior information, which is obtained based on consultation behavior statistics of the doctor on the platform, including “total number of consultation orders”, “Number of Articles”, “a registration number of post-diagnosis patients”, “patient voting”, “thank-you note”, “gift”, “general waiting duration” and “comprehensive recommendation popularity”.

An actual meaning of data is described in Table 1.

TABLE 1
Introduction of Doctor Information Variables

| Variables | Variable Types | Variables Introduction |
|---|---|---|
| Gender | Qualitative Variable | Doctor gender |
| Hospital Level | Qualitative Variable | Hospital level |
| Number of Articles | Quantitative | a number of articles |
| | Variable | about disease |
| | | treatment uploaded |
| | | by a doctor to a |
| | | platform |
| Registration Number | Quantitative | a number of patients |
| of Post-Diagnosis | Variable | to perform a further |
| Patients | | offline consultation |
| | | after the online |
| | | consultation ends |
| Patient Voting | Quantitative | a number of online |
| | Variable | votes for a doctor by |
| | | a patient based on the |
| | | service satisfaction |
| | | degree of the patient |
| | | in a consultation |
| | | process |
| Thank-You Note | Quantitative | a number of thank-you |
| | Variable | notes for a doctor |
| | | written by a patient |
| | | based on the service |
| | | satisfaction degree |
| | | of the patient in a |
| | | consultation process |
| Gift | Quantitative | a number of virtual gifts |
| | Variable | for a doctor by a |
| | | patient based on the |
| | | service satisfaction |
| | | degree of the patient in |
| | | a consultation process |
| General Waiting | Quantitative | a response speed of |
| Duration | Variable | a doctor for patient |
| | | consultation demand |
| Comprehensive | Qualitative | a recommendation popularity |
| Recommendation | Variable | score calculated by |
| Popularity | | a platform based on |
| | | background information, |
| | | patient votes and |
| | | registered patients of a doctor |
| Total number | Quantitative | a total number of orders |
| of orders | Variable | in a platform for |
| | | each doctor in 2019 |
| | | to 2020 based on |
| | | statistics of detailed |
| | | order information, |
| | | which is a prediction |
| | | target of a model |

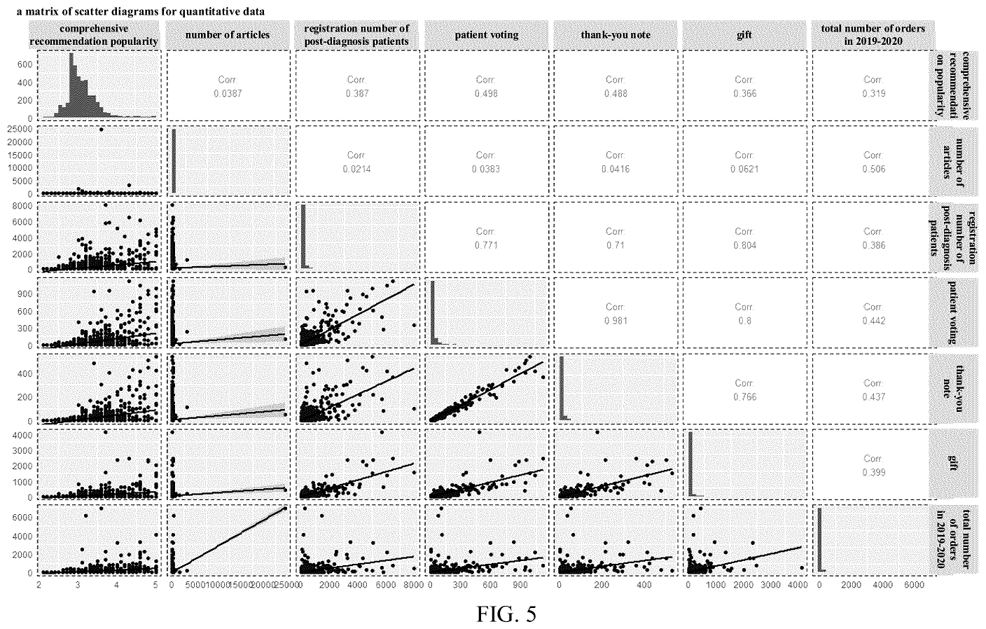

As an example, 5000 pieces of data are extracted, in which 4000 pieces are randomly extracted as training samples, and the remaining 1000 pieces are used as a test set. As illustrated in FIG. 5, for quantitative variables, distribution and correlation of the quantitative variables are displayed in a matrix of scatter diagrams, where each diagram located on a diagonal line of FIG. 5 presents a distribution condition of single data, each diagram located in an upper triangular part presents a correlation coefficient between a pair of variables, and each diagram located in a lower triangular part is a scatter diagram distribution for a pair of variables. In terms of distribution, except for the comprehensive recommendation popularity, other data has a long tailed distribution. As a whole, the quantitative data are index data of platform behaviors, and presents a more obvious positive correlation relationship with each other. The number of articles is close to 0 due to its distribution, and its positive correlations with remaining variables are not significant.

The qualitative variables “gender”, “hospital level” and “general waiting duration”, embodies a doctor composition and a service comfort level of the platform. It can be seen from Table 2 that distribution of male and female proportions in two qualitative variables of a hospital level and a waiting duration are relatively stable; a doctor focuses on the grade 3A hospital, and the waiting time of doctor consultation is mostly “zero”, and it can be seen that a doctor service efficiency in the platform is relatively high.

TABLE 2
Distribution Statistics of Qualitative Variable for Doctor Information
Hospital level

| | Ungraded | Secondary | grade 2A | tertiary | grade 3A | Primary | grade A |
|---|---|---|---|---|---|---|---|
| Gender | hospital | hospital | hospital | hospital | hospital | hospital | hospital |
| Unknown | 0 | 2 | 3 | 11 | 34 | 4 | 0 |
| Male | 70 | 46 | 225 | 296 | 1808 | 40 | 2 |
| Female | 45 | 30 | 120 | 153 | 1085 | 25 | 1 |

General waiting duration

| | Relatively | | | Relatively | | |
|---|---|---|---|---|---|---|
| Gender | slow | Slow | Normal | quick | Quick | None |
| Unknown | 0 | 0 | 2 | 0 | 1 | 51 |
| Male | 28 | 40 | 56 | 54 | 252 | 2057 |
| Female | 15 | 18 | 26 | 30 | 112 | 1258 |

According to an exemplary embodiment of the present disclosure, a total number of orders for doctors in 2019 to 2020 may be predicted by using an XGBoost based on doctor behavior information and registration information (remaining variables except for the total number of orders in Table 1) at the beginning of 2019. According to the above preferred mode, values of different data sets or data points are estimated to address different data problems in the platform, which are described below in several parts.

### A. Effect of Data Set Size on Value

In the art of big data, a larger data volume means a higher accuracy of machine learning. Therefore, accumulation of data is crucial to value accumulation of the platform. In an online platform, a cold start problem occurs due to a large error in an operation strategy since a data volume accumulation of a platform participant is less and the platform has difficulty in data cognition. When the data volume is large enough, precise matching is relatively easy for the platform. However, since the online platform needs to quickly respond to a user request, the data volume may be restricted to a computing power. Therefore, it is important for the platform how to balance values of different data sets and calculation costs.

By using actual data of the online platform, sets with different data volumes are respectively taken, Shapley values of the sets are calculated to measure values of data sets of different sizes and are compared with a classical information entropy (estimated by using Kozahenko-Leonenko). For the platform, the quality of a data set is directly related to a prediction precision MAE. It can be seen that with the increase of the data set size, a prediction error gradually decreases, and a Shapley value gradually increases. That is, a value of the data increases. The Shapley value may depicts that the value increases as the data set size increases. An entropy is a function of a random variable, and is directly determined by its probability distribution. Therefore, entropies of different data sets shall be consistent. However, in actual calculations, it is necessary to estimate the entropy by using a numerical method (Kozahenko-Leonenko), and the increase of the data volume makes its distribution more approximate to a true value, resulting in a decrease of gradual convergence. Therefore, the Shapley value more directly captures a value of the data volume.

### B. Relationship Between Prediction Precision and Actual Platform Utility

In the upper part, the Shapley value successfully captures the value increase brought by the increase of the data volume, so that the prediction precision is improved. In the section, several different training sets are selected to compare a prediction error (MAE) with a platform total revenue (Rₜ) and obtain a relationship.

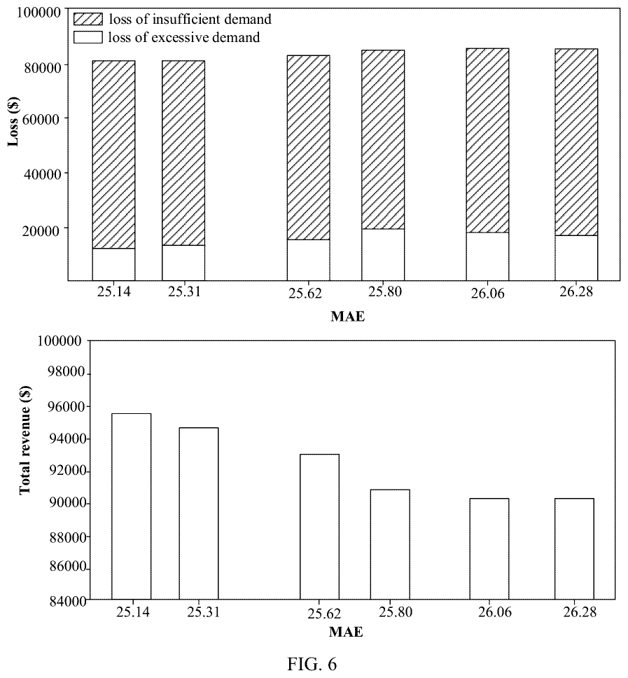

FIG. 6 illustrates a relationship between a platform revenue and a prediction error, a relationship between an extra cost of a platform and a prediction error. As illustrated in FIG. 6, a top picture shows a cost loss caused by a platform operation with increase of the prediction error. The cost is divided into two parts, one of which is an extra employment cost due to insufficient demand, the other of which is a platform processing cost of an excessive demand; and a bottom picture is a relationship between a total revenue of the platform in a single period and the prediction error. The smaller the overall prediction error is, the smaller the extra cost is, and the larger the revenue is.

### C. Interpretation of Prediction Model for Platform Demand

Based on the first two sections, the Shapley value successfully associates the platform revenue with the evaluated data value. In addition, it may be observed how each data point in the model affects the model, that is, how each data point affects the platform revenue, thereby interpreting the model and assisting in formulation of a platform operation strategy.

According to the defined utility function, a Shapley value of each data point in a training set may be calculated. As illustrated in Table 3, an SV_quantile may rank the Shapley values from low to high. There are five groups divided based on each 20% quantile, in which 0%-20% represents a group with the lowest Shapley value, and 80%-100% is a group with the highest Shapley value. Remaining variables in the table 3 represent an average of corresponding values within one group.

Firstly, two extreme groups of data are observed. Compared with other groups of data, the group (80%-100%) of data with the highest “value” is characterized by more gifts and thank-you notes and a higher total number of orders. That is, this conforms to a high correlation between any two variables as shown in FIG. 3. However, in the group of data with a low value (20%-40%), gifts and thank-you notes are low, but a total number of orders is relatively high, and an overall distribution of data does not match.

TABLE 3
Shapley Value and Data Distribution

| | | | | Registration | | |
|---|---|---|---|---|---|---|
| | | Thank- | Comprehensive | Numbers of | Number | Total |
| | | You | Recommendation | Post-Diagnosis | of | number of |
| SV_Quantile | Gift | Note | Popularity | Patients | Articles | orders |
| 0%-20% | −0.036 | −0.066 | −0.052 | −0.037 | 0.067 | 34.6 |
| 20%-40% | −0.040 | −0.041 | −0.057 | −0.046 | −0.022 | 26.0 |
| 40%-60% | −0.058 | −0.041 | −0.049 | −0.059 | −0.019 | 25.6 |
| 60%-80% | 0.093 | 0.052 | 0.024 | 0.084 | −0.014 | 37.1 |
| 80%-100% | 0.040 | 0.095 | 0.134 | 0.058 | −0.011 | 48.5 |

Form 5 groups of data as a whole, it may be seen that two groups of data (20-60%) are relatively approximate, reflecting a basic situation of a large amount of data, and groups of data (60-100%) reflects a data situation that there is a large number of orders. In combination with the above long tailed data distribution, data points with a relatively high total number of orders are correctly predicted, which makes a relatively high contribution to the utility function, and the data value is high. In other words, the platform correctly selects data points with a relatively high demand for the doctor in the actual service are of relatively high value.

In the above model calculation, data with a higher “value” may successfully reflect a positive correlation between each of the quantitative variables (such as a thank-you note and a gift) in the data distribution and an order number. Capturing the correlation helps the platform correctly establish a relationship between doctor data and a service capability of the doctor data. However, low-value data violates the overall data distribution, and generates an interference on a specified strategy of the platform, which reduces the accuracy of the prediction model. Thus, the platform needs to delete the data that generate the interference, so as to ensure the maximum revenue of the platform strategy under the overall samples.

### D. Noise Data Detection

Malicious click farming and comment click farming on the online platform are common problems. Reputations of a doctor accumulated on the platform (such as a number of articles and gifts) are important factors that attract a patient, and such data are deliberately clicked by some doctors. Therefore, demand prediction is distorted, which results in difficulty of platform operation. The Shapley value may recognize the contribution of data in the model, and the user with malicious click farming may have a negative effect on the model, which leads to a lower Shapley value. In this regard, the platform may appropriately delete some low-value data points to improve the quality of data used by the platform.

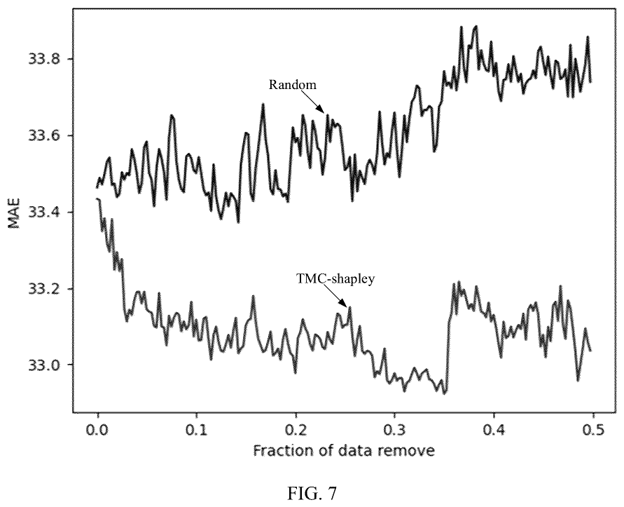

In the experiment of this section, a model effect of deleting low-value data is verified, data points in the training set are permutated based on the Shapley value. Then, a data point is removed in the permutation from low to high, the model is re-trained when one data point is removed each time, and a change of the model effect (MAE, an absolute error between the predicted value and the real value) is observed. The way of removing the data point in the permutation based on the Shapley value is compared with the way of removing the data point in a random sequence, so as to reflect a correction effect of the Shapley value to model prediction. Experimental results are illustrated in FIG. 7, in which a longitudinal axis is the MAE, and a horizontal axis is a fraction of removing data points.

FIG. 7 illustrates a change in a model performance when gradually removing the data point. As a whole, in two ways of removing data, the model performance by removing data based on the data value is better than that by randomly removing data, which indicates that a Shapley concept plays a role in an improvement of the model. From the model performance according to the algorithm in the present disclosure, it may be observed that removing 50% of data within a range may reduce a prediction error. In general, removing data points may make the model performance worse, and it can be seen from the drawings that the loss function is further reduced when data points with lower values are removed in the present disclosure.

Thus, data points with low values affect the overall performance of a model, and it is extremely likely that these data points correspond to some participants of data fraud in the platform. Therefore, it is meaningful that the platform screens out low-value data, which provides high-quality data learning samples for achieving accurate doctor recommendation and doctor patient resource matching in future based on the value calculation of doctor demand data.

In the present disclosure, in a resource allocation scenario of the online medical platform, the value of each data point used by the platform may be effectively measured, and a feasible solution is provided for the platform to improve the operation effect from data to model prediction to platform decision-making.

An apparatus and a method for data processing according to an exemplary embodiment of the present disclosure are described above with reference to FIG. 1 to FIG. 7. It should be noted that the method may be implemented by a program recorded on a computer-readable medium. For example, according to the exemplary embodiments of the present disclosure, a computer medium for data processing based on a data value is provided. The computer program is recorded on the computer-readable medium to performing the method steps of: acquiring at least a portion of high-value data from original data; and performing a service prediction on the acquired high-value data. The value of each piece of original data is calculated based on a utility function associated with a service revenue, and the at least a portion of high-value data are acquired based on a calculation result.

The computer program in the computer-readable medium may be running in an environment deployed in computer devices such as clients, hosts, agent devices, servers, etc. It should be noted that the computer program may also be configured to perform additional steps in addition to the above steps or perform more specific processing when performing the above steps, which are described referring to FIG. 1 to FIG. 7, which may not be repeated here.

It should be noted that the apparatus for data processing according to the exemplary embodiment of the present disclosure may fully rely on the running of the computer program to achieve a corresponding function. That is, each apparatus corresponds to each step in a functional architecture of the computer program, so that the entire system is called by a specialized software package (for example, a lib library) to achieve a corresponding function.

In addition, each apparatus illustrated in FIG. 1 to FIG. 2 may also be implemented by means of a hardware, a software, a firmware, a middleware, a microcode or any combination thereof. When the apparatus is implemented by means of the software, the firmware, the middleware, or the microcode, a program code or a code segment for performing a respective operation may be stored in a computer-readable medium, such as a storage medium, so that the processor may perform the corresponding operation by reading and running the corresponding program code or code segment.

For example, the exemplary embodiment of the present disclosure may also be implemented by a computing device. The computing device includes a storage component and a processor, and a set of computer executable instructions is stored in the storage component. When the set of computer-executable instructions is executed by the processor, a method for data processing according to the exemplary embodiment of the present disclosure is performed.

Specifically, the computing device may be deployed in a server or a client, or may be deployed on a node device in a distributed network environment. In addition, the computing device may be a PC computer, a tablet device, a personal digital assistant, a smartphone, a web application, or other devices capable of executing the instruction set.

The computing device is not necessarily a single computing device, and may be an assembly of any apparatus or circuit capable of executing the instructions (or instruction sets) alone or in combination. The computing device may also be a part of an integrated control system or a system manager, or may be configured as a portable electronic device that is connected with local or remote devices via interfaces (e.g., via wireless transmission).

In the computing device, a processor may include a central processing unit (CPU), a graphics processing unit (GPU), a programmable logic device, a dedicated processor system, a microcontroller or a microprocessor. As an example, the processor may also include an analog processor, a digital processor, a microprocessor, a multi-core processor, a processor array, a network processor, and the like.

Some operations described in the method for data processing according to an exemplary embodiment of the present disclosure may be implemented by a software, or a hardware, or a combination of a software and a hardware.

The processor may run instructions or codes stored in one of storage components. The storage component may also store data. Instructions and data may be also sent and received over a network via a network interface device. The network interface device may employ any known transmission protocol.

The storage component may be integrated with the processor. For example, a RAM or a flash memory may be arranged within an integrated circuit microprocessor. In addition, the storage component may include an independent device, such as an external disk drive, a storage array, or other storage devices that may be used by any database system. The storage component and the processor may be coupled in operations, or may communicate with each other, such as through an I/O port, a network connection, so that the processor may read a file stored in the storage component.

In addition, the computing device may also include a video display (such as a liquid crystal display) and a user interaction interface (such as a keyboard, a mouse and a touch input device). All components of the computing device may be connected to each other via a bus and/or a network.

The operations involved in the method for data processing according to the exemplary embodiment of the present disclosure may be described as various interconnected or coupled functional blocks or functional diagrams. However, the functional blocks or functional diagrams may be equally integrated into a single logic device or operated based on a non-exact boundary.

For example, as described above, the apparatus for data processing based on a data value according to the exemplary embodiment of the present disclosure may include a storage component and a processor. The storage component stores a set of computer executable instructions. When the set of computer executable instructions is executed by the processor, the following steps are performed: acquiring at least a portion of high-value data from original data; and performing a service prediction on the acquired high-value data. The value of each piece of original data is calculated based on a utility function associated with a service revenue, and the at least a portion of high-value data is acquired based on a calculation result.

Exemplary embodiments of the present disclosure are described above, and it should be understood that the above description is merely exemplary and is not intended to be exhaustive, which are not limited in the disclosure. Various modifications and variations are apparent for those skilled in the art without departing from the scope and spirit of the disclosure. Therefore, the protection scope of the present disclosure should be subject to the scope of claims.

What is claimed is:

1. A computer-implemented method for data processing based on a data value, comprising:
calculating a value of each piece of original data based on a utility function associated with a service revenue;
acquiring pieces of high-value data from the original data based on the value of each piece of original data; and
performing a service prediction on the pieces of high-value data.

2. The method of claim 1, wherein performing the service prediction on the pieces of high-value data comprises:
training a service model by using the pieces of high-value data; and
performing the service prediction by using the service model.

3. The method of claim 1, wherein calculating the value of each piece of original data comprises:
calculating a data Shapley value of each piece of original data based on the utility function associated with the service revenue as the value of each piece of original data.

4. The method of claim 3, wherein the utility function is associated with a prediction error of a service model trained by using pieces of the original data.

5. The method of claim 4, wherein:
random permutation on a set of all the original data is performed in each iteration of calculating the utility function; and
in response to a difference between a utility function value of the set of all the original data and a utility function value of a set consisting of any piece of original data and previous elements of any piece of original data being less than a preset threshold, a utility function value of any piece of original data remains unchanged for a set consisting of the previous elements.

6. The method of claim 5, wherein calculating the data Shapley value of each piece of original data comprises:
training a universal set service model based on the set of all the original data, and recording a universal set prediction result of each piece of original data in the universal set service model; and
in each iteration of calculating the utility function, acquiring a prediction result of each piece of original data based on a universal set prediction result of at least one piece of original data close to each piece of original data.

7. The method of claim 1, wherein the original data comprises: at least one period of historical data that occurs before a current moment,
wherein performing the service prediction on the pieces of high-value data comprises:
performing the service prediction for a next period of data based on the pieces of high-value data to obtain prediction data corresponding to the current moment.

8. The method of claim 1, wherein the utility function is adjusted based on at least one of a service crowd, a service logic, an external environment and a time change.

9. The method of claim 1, further comprising:
building a graphical display interface based on the pieces of high-value data to interpret a service model to a user.

10. A computing device, comprising:
a processor; and
a memory stored with instructions executable by the processor;
wherein the processor is configured to:
calculate a value of each piece of original data based on a utility function associated with a service revenue;
acquire pieces of high-value data from original data based on the value of each piece of original data; and
perform a service prediction based on the pieces of high-value data.

11. The computing device according to claim 10, wherein the processor is further configured to:
train a service model by using the pieces of high-value data; and
perform the service prediction by using the service model.

12. The computing device according to claim 10, wherein the processor is further configured to:
calculate a data Shapley value of each piece of original data based on the utility function associated with the service revenue as the value of each piece of original data.

13. The computing device according to claim 12, wherein the utility function is associated with a prediction error of a service model trained by using at least a portion of the original data.

14. The computing device according to claim 13, wherein the processor is further configured to:
perform random permutation on a set of all the original data in each iteration of calculating the utility function; and
in response to a difference between a utility function value of a set consisting of any piece of original data and previous elements of any piece of original data and a utility function value of the set of all the original data being less than a preset threshold, remain a utility function value of any piece of original data unchanged for a set consisting of the previous elements.

15. The computing device according to claim 14, wherein the processor is further configured to:
train a universal set service model based on the set of all the original data, and record a universal set prediction result of each piece of original data in the universal set service model; and
in each iteration of calculating the utility function, acquire a prediction result of each piece of original data based on a universal set prediction result of at least one piece of original data close to each piece of original data.

16. The computing device according to claim 10, wherein the original data comprises at least one period of historical data that occurs before a current moment, and
the processor is further configured to perform the service prediction for a next period based on the pieces of high-value data to obtain prediction data corresponding to the current moment.

17. The computing device according to claim 10, wherein the processor is further configured to:
adjust the utility function associated with the service revenue based on at least one of a service crowd, a service logic, an external environment and a time change.

18. The computing device according to claim 10, wherein the processor is further configured to:
build a graphical display interface based on the pieces of high-value data to interpret a service model to a user.

19. A non-transitory computer readable storage medium having computer programs stored thereon, wherein when the computer programs are executed by a computing device, a method for data processing based on a data value is implemented, the method comprising:
calculating a value of each piece of original data based on a utility function associated with a service revenue;
acquiring pieces of high-value data from original data based on the value of each piece of original data; and
performing a service prediction on the pieces of high-value data.
